(19) United States
(12) Patent Application Publication
(10) Pub. No.: US 2024/0285526 A1
(43) Pub. Date: Aug. 29, 2024

(54) TRANSDERMAL CANNABIS CREAM AND MUSCLE RUB

(71) Applicant: David Radford, Auora, OH (US)

(72) Inventor: David Radford, Auora, OH (US)

(73) Assignee: Day Three Botanicals, LLC, Aurora, OH (US)

(21) Appl. No.: 18/402,579

(22) Filed: Jan. 2, 2024

Related U.S. Application Data
(60) Provisional application No. 63/421,813, filed on Nov. 2, 2022.

(51) Int. Cl. A61K 9/06; A61K 31/00; A61K 47/10; A61K 47/14; A61K 47/36; A61K 47/46

(52) U.S. Cl. CPC A61K 9/06; A61K 31/658; A61K 47/10; A61K 47/14; A61K 47/36; A61K 47/46

(57) ABSTRACT

The present disclosure relates to a topical transdermal cream and muscle rub formulated to address both skin pain and itch due to inflammation and other problems. The transdermal cream is formulated by components including: cannabidiol (CBD), gamma amino butyric acid (GABA), at least one terpenoid, at least one flavonoid, a penetrant, and at least one emollient. The CBD and GABA components work by perpetrating skin layers and interacting with various receptors in the human body, therefore providing relief. A method of making the topical transdermal cream using the claimed components is also disclosed herein.

### BACKGROUND DESCRIPTION OF THE INVENTION

### 1. Field of the Invention

The present invention is a topical transdermal multimodal cream developed to address both dermal (skin) pain and itch. The need for topical cream able to address both dermal pain and itch sensations without a prescription is greatly needed. Most over-the-counter topical skin preparations generally use counterirritants to address pain and they do not suffice when there is associated inflammation or itching.

### 2. Background of the Invention

Both Gabapentin and Pregabalin are products that require a prescription. Gamma amino butyric acid or GABA on the other hand GABA is a non-essential, hydrophilic, amino acid. GABA or GABA residues can bind in Ligand-gated ion channels reducing the presynaptic response. (Lummis SC. Locating GABA in GABA receptor binding sites. Biochem Soc Trans. 2009 December;37(Pt 6):1343-6. doi: 10.1042/BST0371343. PMID: 19909273.)

Compounds containing Delta-9-tetra-hydrocannabinol (THC), a psychoactive cannabinoid found in the plants Cannabis Sativa, Cannabis Indica and Cannabis Ruderalis. The use of THC is restricted by law according to the United States Drug Enforcement Agency, because the substance (THC) has a high potential for abuse, it has no currently accepted medical treatment indications in the U.S.

Cannabidiol (CBD) is a molecule similar in shape to THC, it is not a Schedule 1 substance like THC. CBD is regulated under Agriculture Improvement Act of 2018. The provisions of the “Farm Bill” state that the CBD must be derived from the hemp plant (Cannabis sativa L.) and products made from plant material must contain less than 0.3% THC (tetrahydrocannabinol) by dry weight. The phytogenic CBD in this discussion is an isolate from the Cannabis sativa L plant, and it is free from THC.

Both CBD and THC are cyclic compounds, meaning one or more series of atoms in the composition connect to form a ring. Both molecules have the exact same atom composition: 21 carbon atoms, 30 hydrogen atoms, and 2 oxygen atoms. The molecules in CBD are arranged in an open ring (hydroxyl group), while THC is arranged in a closed (cyclic) ring, and as a result of the molecular shape, CBD does not have psychogenic properties like THC.

This simple open versus closed ring arrangement changes the way these cannabinoids interact with the cannabinoid receptors. Ligands like THC and CBD bind on specific receptors in the endocannabinoid system (ECS) and produce specific responses. Three such receptors have been discovered and their locations have been described, providing a pathway for means of administration.

The skin is the body's largest organ system and makes up to 15% or more of an individual's weight. Skin is a barrier between an individual and their environment. Both synthetic and natural substances can be absorbed by the skin. The human skin is able to resist the penetration of many molecules. In particular, large molecules are not easily absorbed. There is a 500 Dalton rule for the skin penetration of chemical compounds and drugs. The 500-Dalton rule states that for a substance to be able to cross the skin barrier, it must have a molecular weight of fewer than 500 Daltons. However, especially smaller molecules can be absorbed transcutaneously, some with the assistance of substances like penetrants that breakdown the natural resistance of skin to allow enhanced and more rapid absorption. Absorption of a substance through the skin depends on a number of factors: concentration, molecular weight and size of the molecule, the duration of contact with the substance, the solubility of the substance, as well as the physical condition of the skin. Substances applied to skin, like cleansers are typically removed in a short time, whereas substances like emollients are applied to the skin and are absorbed or evaporate.

Medical use of cannabis and associated phyto cannabinoids is becoming widely accepted in the United States and Canada as an alternative form of medicine. Many states have legalized its use for qualified medical conditions such as chronic pain, epilepsy, sleep disorders, anxiety, cancer, glaucoma, nausea, ALS, Alzheimer's disease, Crohn's disease, Post-traumatic Stress Disorder (PTSD), arthritis, fibromyalgia, and others. There are well known prior transdermal delivery systems that utilize alcohol, solvents, glycols, heat, and DMSO (dimethylsulfoxide). Some of these systems may also irritate, dehydrate, damage the skin, as well as potentially exhibit various toxic side effects internally.

### SUMMARY OF THE INVENTION

A transdermal cannabis comfort cream and muscle rub (Canna) is formulated in a base. The resulting preparation is an emollient that soothes and moisturizes dry skin, preparing the skin for absorption of two key active ingredients in the formulation: cannabidiol (CBD) and gamma amino butyric acid (GABA). The preparation contains penetrants like Mentha piperita (Peppermint) Oil to aid in transdermal delivery of the active ingredients CBD and GABA. The formulation may also contain terpenoids as well. The CBD acts as a natural anti-inflammatory, anti-bacterial, and antifungal in synergy with terpenophenolic compounds that provide a medicinal effect without any centralized psychotropic effect. Complementary terpenoids and flavonoids provided by a Cymbopogon citratus (Lemongrass) aqueous extract and a Butyrospermum parkii (Shea) leaf aqueous extract provide an entourage (synergistic) effect. GABA serves to block pathological presynaptic pain in the free nerve endings in skin while sparing non-acute TRPV1 pain signals.

The transdermal cannabis comfort cream and muscle rub of this disclosure provides a method of treating the skin and underlying muscle in a subject by applying the cream topically to the skin of the subject, thereby producing anti-inflammatory, anti-bacterial, and antifungal effects in the skin and underlying muscle.

This amphiphilic emulsion is a compounded combination of GABA and phytogenic CBD, along with emulsifiers and surfactants. This novel mixture results in a topical transdermal cream that can address both superficial dermatomal pain and itch response with an over-the-counter product.

Plants have been used as medicine since ancient times. With the aid of science and technology, plants and plant extracts are an increasing subject of scientific investigation, and therapeutic applications of phytogenic compounds are being studied in greater detail as an alternative to synthetic substances today. CBD was first extracted from Cannabis sativa, a discovery attributed to a Harvard trained chemist named Roger Adams, doing research at the University of Illinois in 1940, soon after he discovered THC. Today CBD, a phytocannabinoid, is extracted by various means from the buds of the species Cannabis sativa L, where it is in far more abundant in concentration than THC.

There has been an outpouring of research into studying the various species of plants in the family called Cannabaceae which also includes hops. Each the three major varieties of Cannabis, Sativa, Indica and Ruderalis have different ratios of the 7 major cannabinoids, including CBD and THC, along with many associated compounds.

Through the process of time and evolution, cultivation and hybridization, the chemical profiles of plants can be modified, and through cloning they can be fixed into Chemotypes and Chemovars with predictable CBD to THC ratios, as well as the bioavailability and presence of certain organic compounds known as terpenes. Terpenes are aromatic compounds found in many plants and are often associated with cannabis. Terpenes are believed to facilitate cannabinoid absorption and offer unique therapeutic properties of their own including enhancing the effects of CBD and THC, a process that is called the entourage effect.

### DETAILED DESCRIPTION OF THE INVENTION

The present invention is a topical preparation for treating inflammation, itching, and other ailments of the skin. The preparation is a Topical Allosteric Emulsion (base) with Cannabidiol (CBD), Gamma Amino Butyric Acid (GABA) Transdermal Base Ingredients and Emulsifiers, the CBD and GABA being the primary or active ingredients with emulsifiers, preservatives, emollients, moisturizers, etc. as partially listed below. Barbadensis leaf juice (organic Aloc), Aqua (deionized water) Helianthus annus seed oil (organic), Isopropyl Palmitate, Glyceryl stearate, Cetyl Alcohol, Steric acid, Glycerin (vegetable/kosher), Phenoxyethanol (preservative), Butyrosperum parkii butter (Shea butter), Simmondsia chinensis seed oil (organic Jojoba), Tocopheryl acetate (vitamin E), Xanthan gum (emulsifier), Panthenol (vitamin B-5), Camellia sinensis leaf extract (green tea), and Ethylhexylglycerin (preservative). Complementary terpenoids and flavonoids provided by a Cymbopogon citratus (Lemongrass) aqueous extract and a Butyrospermum parkii (Shea) leaf aqueous extract provide an entourage (synergistic) effect. The preparation contains penetrants like Mentha piperita (Peppermint) Oil to aid in transdermal delivery of the active ingredients CBD and GABA.

Somatosensory stimulus to the skin caused by either a mechanical force, a temperature change, tissue damage, or a chemical action, activating the cells that provide sensory input, can be modulated by a novel amphiphilic emulsion with a compounded combination of gamma butyric acid, cannabidiol and carriers.

In biochemistry and physiology, a ligand is any molecule or atom which binds reversibly to a protein. A ligand can be an individual atom or ion. It can also be a larger and more complex molecule made from many atoms. A ligand can be natural, as an organic or inorganic molecule. Ligands can also be made synthetically, in the laboratory. This is because the key properties of a ligand are found in its chemical structure. If that structure can be recreated in the laboratory, the synthetic ligand will be able to interact in the same way as a naturally occurring ligand.

A ligand that can bind to and alter the function of the receptor that triggers a physiological response is called a receptor agonist. Ligands that bind to a receptor but fail to activate the physiological response are receptor antagonists.

The phyto or plant derived cannabinoids are unique in that they can interact with the endocannabinoid system (ECS) in vertebrates, including human beings. The endocannabinoid system in vertebrates binds with endogenous compounds called Anandamide and AG-2 or 2-Arachidonoylglycerol, they are both endogenous agonists derived from omega fatty acids to bind on vertebrate endocannabinoid receptors in the ECS. CBD derived from Cannabis sativa, is biologically active in vertebrates through a phenomenon called molecular mimicry, allows these plant molecules to bind as ligands with the vertebrate ECS receptor sites.

These receptors sites consist of complex proteins on the cell membrane called G coupled receptors. G protein-coupled receptors (GPCR), also called seven-transmembrane receptor or heptahelical receptors. G protein-coupled receptors (GPCRs) are important cell signaling mediators, involved in essential physiological processes. GPCRs respond to a wide variety of ligands from light to large macromolecules. The GPCR family is characterized by its seven transmembrane spanning domains and represents the largest class of drug targets. There are more than 800 known GPCRs in the human genome, which are involved in virtually every physiological process. (Roux, B.T .; Cottrell, G.S. G Protein-Coupled Receptors: What a Difference a ‘Partner’ Makes. Int. J. Mol. Sci. 2014, 15, 1112-1142. https://doi.org/10.3390/ijms15011112)

Human skin is made up of two basic layers, the outer most layer is the epidermis, and the inner most layer, the dermis. When a topical is applied to the outermost layer, the compounding of this amphiphilic emulsion provides for its absorptive properties. In accordance with the present invention, a compounded combination of GABA and CBD, along with emulsifiers and surfactants is applied directly to the skin, where it is absorbed by the epidermis and makes its way through the 4 outermost layers of skin dells, migrating by absorption until it reaches the dermis. The dermis is the deepest layer of skin. It is more complex than the epidermis, which is mainly a protective barrier. These four layers of the epidermis include the stratum corneum, the keratinized outermost layer which is mostly made up of dead skin cells that have migrated up from the second layer, the stratum granulosum. The stratum granulosum is the second barrier in this epidermal layer, it helps the skin prevent moisture loss, it contains keratinocytes are called granular cells, and contain keratohyalin and lamellar granules. Directly beneath the stratum granulosum is the stratum Spinosum. This epidermal layer it is also referred to as the “spinous” or “prickle-cell” layer This reference is distinguished in its appearance by the microscopic feature of cell-to-cell adhesion of adjacent cells, providing added strength to the epithelial tissuc. The deepest layer of the epidermis is the stratum germinatum, as its name implies, it provides the germinal cells necessary for the continuous regeneration of the three outer layers of the epidermis.

Human skin contains the furthest extension of the nervous system and a primary sensor for harmful and innocuous external stimuli. Skin is not just a protective covering, it is also the largest sensory organ. Unique among human sensory systems, the skin's nervous system gives rise to distinct sensations, including gentle touch, pain, itch, warmth, and cold. These distinct perceptions are initiated by an impressive array of somatosensory neurons, whose sensory axons, called afferents, densely innervate the skin. Humans rely on sensory inputs from the skin to interact with objects in their environment and to avoid harm. (Owens DM, Lumpkin EA. Diversification and specialization of touch receptors in skin. Cold Spring Harb Perspect Med. 2014 Jun. 2;4(6):a013656. doi: 10.1101/cshperspect.a013656. PMID: 24890830; PMCID: PMC4031957.)

Once the inventive formula penetrates the epidermis, the amphiphilic emulsion containing phytogenic CBD, GABA and terpenes pass through the skin, these ligands can then travel through the watery fluids of the user within the blood stream, dispersed via the tissues, to the wall of the cell itself. As the amphiphilic emulsion is dispersed locally, the ligands are in direct contact with the specialized tissues, these including the sweat glands, hair roots, oil glands, blood vessels, lymphatics, target GPCRs and ion channels of the various somatosensory neurons involved in nociception. The perception of external stimuli can include the sense of touch (via mechanoreceptors), pain (via nociceptors), and temperature (via thermal receptors). The four major types of tactile mechanoreceptors include: Merkel's disks, Meissner's corpuscles, Ruffini endings, and Pacinian corpuscles. The encoding of the mechanical force is attributed to three types of mechanoreceptors reacting to distinct intensity ranges. These include T cells for touch, P cells for pressure, and N cells for strong, noxious skin stimulation.

The target for the phytogenic CBD molecule is the GPCRs (the cannabinoid receptors in skin) which include the cannabinoid receptor-2 (CB2) receptor and the more recently discovered TRP channels. It is believed that activation of CB2 receptor results in β-endorphin release from keratinocytes, which then acts on primary afferent neurons to inhibit nociception. The CB2 receptor is mainly expressed peripherally, with CB2 receptors in immune tissues, highlighting the possibility that the ECS has an immunomodulatory role. In this respect, the CB2 receptor was shown to modulate immune cell functions in animal and human models of inflammatory diseases. (Turcotte C, Blanchet MR, Laviolette M, Flamand N. The CB2 receptor and its role as a regulator of inflammation. Cell Mol Life Sci. 2016 December;73(23):4449-4470. doi: 10.1007/s00018-016-2300-4. Epub 2016 Jul. 11. PMID: 27402121; PMCID: PMC5075023.)

Transient receptor potential (TRP) channels are a group of membrane proteins involved in the transduction of a plethora of chemical and physical stimuli. These channels modulate ion entry, mediating a variety of neural signaling processes implicated in the sensation of temperature, pressure, and pH, as well as smell, taste, vision, and pain perception. Many diseases involve TRP channel dysfunction, including neuropathic pain, and inflammation. In the pursuit of new treatments for these disorders, it was discovered that cannabinoids can modulate a certain subset of TRP channels. The TRP vanilloid (TRPV), TRP ankyrin (TRPA), and TRP melastatin (TRPM) subfamilies were all found to contain channels that can be modulated by several endogenous, phytogenic, and synthetic cannabinoids. To date, six TRP channels from the three subfamilies mentioned above have been reported to mediate cannabinoid activity: TRPV1, TRPV2, TRPV3, TRPV4, TRPA1, and TRPM8. The increasing data regarding cannabinoid interactions with these receptors has prompted some researchers to consider these TRP channels to be “ionotropic cannabinoid receptors.” Although CB1 and CB2 are the canonical cannabinoid receptors, there is significant overlap between cannabinoids and ligands of TRP receptors. (Muller C, Morales P, Reggio PH. Cannabinoid Ligands Targeting TRP Channels. Front Mol Neurosci. 2019 Jan. 15;11:487. doi: 10.3389/fnmol.2018.00487. PMID: 30697147; PMCID: PMC6340993.)

The skin is rich in cannabinoid receptors, in both structural and immune cells, and sensory nerve endings. Support of cannabinoid receptors leads to therapeutic application of exogenous (external) cannabinoids in the regulation and preservation of the skin and its functionality.

The somatosensory system informs the human body about objects in our external environment through touch (i.e., physical contact with skin) and about the position and movement of our body parts (proprioception) through the stimulation of muscle and joints. The somatosensory systems also monitor the temperature of the body, external objects, environment, and provides the brain with information about painful, itchy and tickling stimuli. The sensory information processed by the somatosensory systems travels along different anatomical pathways depending on the information carried. For example, the posterior column-medial lemniscal pathway carries both discriminative touch and proprioceptive information from the body, and the main sensory trigeminal pathway carries this information from the face. Whereas, the spinothalamic pathways carry crude touch, pain and temperature information from the skin via primary afferent neurons (A and C fibers), and the spinal trigeminal pathway carries this same information from the face.

There is a vast array of TRP Channel Cannabinoid Receptors involved in skin sensation., In the skin, cannabinoid lipids, whether of endogenous or exogenous origin, can regulate numerous sensory, homeostatic, and inflammatory events. Although many of these effects are mediated by metabotropic cannabinoid receptors, a growing body of evidence has revealed that multiple members of the transient receptor potential (TRP) ion channel family can act as “ionotropic cannabinoid receptors”. Furthermore, many of these same TRP channels are intimately involved in cutaneous processes that include the initiation of pain, temperature, and itch perception, the maintenance of epidermal homeostasis, the regulation of hair follicles and sebaceous glands, and the modulation of dermatitis. Ionotropic cannabinoid receptors therefore represent potentially attractive targets for the therapeutic use of cannabinoids to treat sensory and dermatological diseases. (Caterina MJ. TRP channel cannabinoid receptors in skin sensation, homeostasis, and inflammation. ACS Chem Neurosci. 2014 Nov. 19;5(11):1107-16. doi: 10.1021/cn5000919. Epub 2014 Jun. 17. PMID: 24915599; PMCID: PMC4240254.) In the skin, the cannabinoid lipids, whether endogenous or exogenous in origin, function as adaptogens, as they can regulate numerous sensory, homeostatic, and inflammatory events.

Once applied as a transdermal, both the CBD and GABA ligands travel at random, once their concentration is high enough, a ligand will eventually reach the surface membrane G protein-coupled receptors (GPCRs). Proteins receiving ligands can be receptors or ion channels and can even be the start of a complex series of intertwined proteins. When a ligand binds to the protein receptor, it undergoes a conformational change. This means that while no chemical bonds have been formed or broken, and the change is reversable, the physical action of the ligand fitting into the protein receptor temporarily changes the overall shape of the entire structure.

This change in shape can trigger many physiological actions. In most cases, the movement of the protein itself activates another chemical pathway, or triggers the release of another messenger ligand, to carry the message to other receptors. This reversibility of the bond between ligands like CBD and a vast number of proteins are a crucial aspect of all forms of life.

Among the principal ingredients in the inventive preparation are cannabidiol (CBD) and gamma amino butyric acid (GABA). The preparation is derived from an aqueous solution in which hydrocarbon-based surfactants are compounded into an amphiphilic mixture, having both a hydrophilic or water interactive “end”, referred to as the “head group” that bind water to the GABA molecule, and a fat soluble or lipophilic “end” that bind the lipid to the CBD molecule. The result is a homogenous mixture of hydrophilic and lipophilic residues.

CBD has emerged as a promising naturally occurring compound being used in the treatment of several diseases. Its diverse pharmacological effects with limited side effects offer several potentially safer therapeutic applications. The CBD molecule is hydrophobic and lipophilic, meaning that it does not dissolve, or be emulsified in water, but it will dissolve in fat as well as most organic solvents. To effectively administer CBD transdermally, the ligand needs to be incorporated into a hypogenic, amphiphilic emulsion that can be applied directly to the skin. Once in tissue, the CBD can begin its work as an adaptogen, while the GABA modulates ion channels.

The presence of GABA in ionic form in the amphiphilic emulsion starts a reaction that makes it less likely that a presynaptic action potential will be passed antidromically on to other neurons. GABA(A) receptors are chloride permeable, g-amino butyric acid (GABA)-gated ion channels that are responsible for the majority of fast inhibitory neurotransmission in the mammalian nervous system. This presynaptic inhibition, an increased conductance in the membrane of the presynaptic neuron is presumed to reduce the action potential, thereby reducing transmitter release. (Graham B, Redman S. A simulation of action potentials in synaptic boutons during presynaptic inhibition. J Neurophysiol. 1994 February;71(2):538-49. doi: 10.1152/jn. 1994.71.2.538. PMID: 8176423.)

GABA is classified biologically as a ligand, GABA and its residues can bind in Ligand-gated ion channels (LICs, LGIC) or inotropic receptors. GABA is considered in fast synaptic inhibition. Upon the receptor binding to GABA, a chloride ion-selective pore opens to allow chloride to move across the cell membrane. This influx of chloride reduces the electrical potential, acting as an inhibitor of presynaptic neuronal action potential. (Allen MJ, Sabir S,

Sharma S. GABA Receptor. [Updated 2022 Feb. 17]. In: StatPearls [Internet]. Treasure Island (FL): StatPearls Publishing; 2022 January-. Available from: https://www.ncbi.nlm.nih.gov/books/NBK526124/)

Ligand-gated ion channels (LICs, LGIC), also commonly referred to as ionotropic receptors, are a group of transmembrane ion-channel proteins which open to allow ions such as Sodium, Potassium, Calcium, and/or Chloride to pass through the membrane in response to the binding of a chemical messenger (i.e., a ligand), such as a neurotransmitter like GABA.

When a presynaptic neuron is excited, it releases a neurotransmitter from vesicles into the synaptic cleft. The neurotransmitter then binds to receptors located on the postsynaptic neuron. If these receptors are ligand-gated ion channels, a resulting conformational change opens the ion channels, which leads to a flow of ions across the cell membrane. This, in turn, results in either a depolarization, for an excitatory receptor response, or a hyperpolarization, for an inhibitory response.

These receptor proteins are typically composed of at least two different domains: a transmembrane domain which includes the ion pore, and an extracellular domain which includes the ligand binding location (an allosteric binding site). The function of such receptors located at synapses is to convert the chemical signal of a presynaptically released neurotransmitter directly and very quickly into a postsynaptic electrical signal.

In ionotropic GABA(A) receptors, binding of GABA molecules to their binding sites in the extracellular part of the receptor triggers opening of a chloride ion-selective pore.

Consequently, CBD works by interacting with the numerous cannabinoid receptors of our body's system. The cannabinoid receptor type 2, abbreviated as CB2, is a G protein-coupled receptor from the cannabinoid receptor family that in humans is encoded by the CNR2 gene.

A user can apply the inventive preparation directly on the skin with an applicator to penetrate the skin layers and interact with the body's endocannabinoid receptors for the intended relief.

The administration of an allosteric transdermal cream with fixed combinations of CBD and GABA is a novel approach in both the skin penetration of the cargo compounds and their synergistic effects. The cutaneous administration of cannabidiol (CBD) and gamma amino butyric acid (GABA) in a fixed combination can be useful for the localized treatment of neuropathic pain, regional inflammation, itch and dry skin. The inventive preparation is a hydrophilic-lipophilic balanced formulation of biocompatible microemulsions as the vehicle for skin delivery (by way of topical application) of cannabidiol and gamma amino butyric acid. The ingredients in the various combinations as listed below are mechanically homogenized, starting with the active ingredients cannabidiol (CBD) and gamma amino butyric acid (GABA), which are mixed into a milky nanoemulsion. Biosurfactants are added to the nanoemulsion during homogenization, so that when it is applied to skin, it is readily absorbed. Peppermint oil in particular is known to enhance absorption through the skin. Anna Hermana and Andrzej P. Herman, 2014 Royal Pharmaceutical Society, Journal of Pharmacy and Pharmacology, 67, pp. 473-485. In a key aspect of the invention, the base ingredients cannabidiol (CBD) and gamma amino butyric acid (GABA) are a hydrophilic-lipophilic balanced formulation of biocompatible microemulsion that is mechanically homogenized under low pressure into a nanoemulsion. The additional ingredients may be varied as noted in the Table below.

Table 1 is a list of percentages of ingredients in the transdermal pharmaceutical composition of the invention. To make the preparation, once the total desired weight of the finished preparation is determined, a quantity of water equal to 52 to about 89% w/w is put into a mixing vessel, the CBD and GABA added at between .5 and 1.0% w/w and mixed until a milky nanoemulsion is formed. The ingredients as listed in the Table are then added to the nanoemulsion in their respective percentages and mixed until uniform in the well known manner.

TABLE 1

| Ingredient | W/W % Range | |
|---|---|---|
| Water | 52.000 | 88.999 |
| Aloe Barbadensis Leaf Juice | 5.0000 | 10.000 |
| Isopropyl Palmitate | 1.0000 | 5.0000 |
| Helianthus Annuus (Sunflower) Seed Oil | 1.0000 | 5.0000 |
| Glyceryl Stearate SE | 1.0000 | 5.0000 |
| Cetyl Alcohol | 1.0000 | 5.0000 |
| Stearic Acid | 1.0000 | 5.0000 |
| Glycerin | 1.0000 | 5.0000 |
| GABA | 0.5000 | 1.0000 |
| Cannabidiol (CBD) | 0.5000 | 1.0000 |
| Mentha Piperita (Peppermint oil) | 0.0150 | 0.1500 |
| Phenoxyethanol | 0.0001 | 1.0000 |
| Xanthan Gum | 0.0001 | 1.0000 |
| Butyrospermum Parkii (Shea) Butter | 0.0001 | 1.0000 |
| Simmondsia Chinensis (Jojoba) Seed Oil | 0.0001 | 1.0000 |
| Panthenol | 0.0001 | 1.0000 |
| Tocopheryl Acetate | 0.0001 | 1.0000 |
| Camellia Sinensis Leaf Extract | 0.0001 | 1.0000 |
| Ethylhexylglycerin | 0.0001 | 1.0000 |
| Cannabigerol (CBG) | 0.0000 | 1.0000 |

It will be understood that various changes in the details and materials which have been described and illustrated above in order to explain the nature of this invention may be made by those skilled in the art without departing from the principle and scope of the invention as recited in the following claims.

### REFERENCES RELATED TO THE INVENTION

https://patents.justia.com/patent/20200016115
https://patents.justia.com/patent/20200085959
https://patents.google.com/patent/US20,200,016115A1/en
https://patents.google.com/patent/US10,933,082B2/en?q=cbd+pain+cream&oq=cbd+pain+cream
https://patents.google.com/patent/CA2938621C/en?q=cbd+pain+cream&oq=cbd+pain+cream
https://www.freepatentsonline.com/y2020/0016115.html

1. A topically applied pharmaceutical composition comprising:
cannabidiol, gamma amino butyric acid, at least one terpenoid, at least one flavonoid, a penetrant, and at least one emollient.

2. The composition of claim 1 wherein said penetrant is peppermint oil.

3. The composition of claim 1 wherein said at least one emollient is Isopropyl Palmitate.

4. The composition of claim 1 wherein said composition includes one or more moisturizers.

5. The composition of claim 4 wherein one of said moisturizers is glycerin.

6. The composition of claim 4 wherein one of said moisturizers is xanthan gum.

7. The composition of claim 1 wherein said at least one emollient has one or more of antibacterial, anti-itch, anti-inflammation, or anti-fungal properties.

8. The composition of claim 1 wherein said composition comprises between 0.5 and 1% w/w of cannabidiol and between 0.5 and 1% w/w of gamma amino butyric acid.

9. A method of making a pharmaceutical composition comprising the steps of:
adding CBD and GABA to a quantity of water and mixing until a nanoemulsion is formed; and,
adding at least one terpenoid, at least one flavonoid, a penetrant, and at least one emollient to said nanoemulsion and mixing until a homogenous state is reached.

10. The method of claim 9 wherein said quantity of water is between 52 and 89% w/w and said CBD and GABA are each between 0.5 and 1% w/w.

11. The composition of claim 9 wherein said penetrant is peppermint oil.

12. The composition of claim 9 wherein said at least one emollient is Isopropyl Palmitate.

13. The composition of claim 9 wherein said composition includes one or more moisturizers.

14. The composition of claim 13 wherein one of said moisturizers is glycerin.

15. The composition of claim 13 wherein one of said moisturizers is xanthan gum.

16. The composition of claim 9 wherein said at least one emollient has one or more of antibacterial, anti-itch, anti-inflammation, or anti-fungal properties.
